(19) United States
(12) Patent Application Publication
(10) Pub. No.: US 2023/0170097 A1
(43) Pub. Date: Jun. 1, 2023

(54) METHODS AND COMPUTING DEVICE RELATED TO SLEEP EVALUATION

(71) Applicant: National Yang Ming Chiao Tung University, Taipei City (TW)

(72) Inventor: Albert Chihchieh Yang, Taipei City (TW)

(73) Assignee: National Yang Ming Chiao Tung University, Taipei City (TW)

(21) Appl. No.: 17/889,692

(22) Filed: Aug. 17, 2022

(30) Foreign Application Priority Data
Nov. 29, 2021 (TW) 110144350

(51) Int. Cl. G16H 50/70; G16H 10/20

(52) U.S. Cl. CPC G16H 50/70; G16H 10/20

(57) ABSTRACT

A computing device adapted for performing a method for establishing at least one sleep-related evaluation model and a method for sleep evaluation. The computing device includes a storage unit and a processing unit. The storage unit is configured to store a sleep evaluation model that the processing unit establishes based on multiple pieces of training answer information related to a sleep-related questionnaire and by performing the method for establishing at least one sleep-related evaluation model. The processing unit is configured to perform the method for sleep evaluation, in which the sleep evaluation model thus established and stored is utilized, to detect and recognize dyssomnia based on a piece answer information related to a respondent.

### CROSS-REFERENCE TO RELATED APPLICATION

This application claims priority of Taiwanese Invention Patent Application No. 110144350, filed on Nov. 29, 2021.

### FIELD

The disclosure relates to sleep evaluation, and more particularly to methods for establishing at least one sleep-related evaluation model and for utilizing the at least one sleep-related evaluation model to perform sleep evaluation, and at least one computing device implementing the methods.

### BACKGROUND

As the connection between sleep quality and physical and mental health becomes more and more noticeable to the general public, sleep evaluation becomes an important issue. Sleep evaluation may be performed to detect sleep disorders such as insomnia and sleep apnea. A conventional method for sleep evaluation and estimation of the sleep disorders highly relies on human consultation, and is consumptive in terms of human resources and time.

### SUMMARY

Therefore, an object of the disclosure is to provide at least one method and at least one computing device that can alleviate at least one of the drawbacks of the prior art.

According to one aspect of the disclosure, a method for establishing at least one sleep-related evaluation model is to be performed by a computing device that stores multiple pieces of training answer information which are respectively related to multiple respondents and which are related to a sleep-related questionnaire including multiple questions. Each of the pieces of training answer information includes multiple answers that are respectively related to the questions and that are each related to at least one of multiple evaluation subjects. The multiple evaluation subjects include a particular evaluation subject. Each of the pieces of training answer information is associated with a classification result that is one of insomnia and sleep apnea. The method includes determining multiple first training data sets that correspond respectively to the pieces of training answer information by, with respect to each of the pieces of training answer information: retrieving the answers of the piece of training answer information; with respect to each of the multiple evaluation subjects, determining a training index value based on those of the answers that are related to the evaluation subject; and collecting the training index values thus determined for the multiple evaluation subjects, the classification result associated with the piece of training answer information, and those of the answers that are related to the particular evaluation subject to form the first training data set that corresponds to the piece of training answer information. The method further includes using the first training data sets to train a machine learning model, in order to establish a sleep evaluation model.

According to one aspect of the disclosure, a method for sleep evaluation is to be performed by a computing device that stores a sleep evaluation model which is established according to the method for establishing at least one sleep-related evaluation model. The method for sleep evaluation includes steps of: obtaining a piece of answer information that is related to a respondent and a sleep-related questionnaire, wherein the sleep-related questionnaire includes multiple questions, and the piece of answer information includes multiple answers respectively related to the questions; determining a sleep quality score based on the piece of answer information thus obtained; determining whether dyssomnia is detected based on the sleep quality score thus determined; and when dyssomnia is detected, using the sleep evaluation model to determine a classification result with respect to the respondent based on the piece of answer information, wherein the classification result is one of insomnia and sleep apnea.

According to one aspect of the disclosure, a computing device includes a storage unit and a processing unit that is electrically connected to the storage unit. The storage unit stores multiple pieces of training answer information that are respectively related to multiple respondents and that are related to a sleep-related questionnaire including multiple questions. Each of the pieces of training answer information includes multiple answers that are respectively related to the questions and that are each related to at least one of multiple evaluation subjects. The multiple evaluation subjects include a particular evaluation subject. Each of the pieces of training answer information is associated with a classification result that is one of insomnia and sleep apnea. The processing unit is configured to determine multiple first training data sets that correspond respectively to the pieces of training answer information stored in the storage unit by, with respect to each of the pieces of training answer information: retrieving the answers of the piece of training answer information; with respect to each of the multiple evaluation subjects, determining a training index value based on those of the answers that are related to the evaluation subject; and collecting the training index values thus determined for the multiple evaluation subjects, the classification result associated with the piece of training answer information, and those of the answers that are related to the particular evaluation subject to form the first training data set that corresponds to the piece of training answer information. The processing unit is further configured to use the first training data sets to train a machine learning model, in order to establish a sleep evaluation model.

### BRIEF DESCRIPTION OF THE DRAWINGS

Other features and advantages of the disclosure will become apparent in the following detailed description of the embodiment (s) with reference to the accompanying drawings, of which:

FIG. 1 is a block diagram that exemplarily illustrates a computing device according to an embodiment of the disclosure;

FIG. 2 is a flow chart that exemplarily illustrates a first procedure of a method for establishing at least one sleep-related evaluation model according to an embodiment of the disclosure;

FIG. 3 is a flow chart that exemplarily illustrates sub-steps of Step 21 according to an embodiment of the disclosure;

FIG. 4 is a flow chart that exemplarily illustrates a second procedure of the method according to an embodiment of the disclosure;

FIG. 5 is a flow chart that exemplarily illustrates sub-steps of Step 41 according to an embodiment of the disclosure;

FIG. 6 is a flow chart that exemplarily illustrates a third procedure of the method according to an embodiment of the disclosure;

FIG. 7 is a flow chart that exemplarily illustrates a method for sleep evaluation according to an embodiment of the disclosure;

FIG. 8 is a flow chart that exemplarily illustrates sub-steps of Step 702 according to an embodiment of the disclosure; and

FIG. 9 is a flow chart that exemplarily illustrates sub-steps of Step 705 according to an embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

FIG. 1 exemplarily illustrates a computing device according to an embodiment of the disclosure. The computing device includes a storage unit 1, an output unit 2, and a processing unit 3 that is electrically connected to the storage unit 1 and the output unit 2. According to some embodiments, the computing device may be, for example, a personal computer (PC), a notebook, a tablet computer or a smart phone. The storage unit 1 may be, for example, random access memory (RAM) , read only memory (ROM), programmable ROM (PROM) or flash memory. The processing unit 3 may be a central processing unit (CPU), for example. The output unit 2 may be a display, for example. The computing device may further include an input unit (not shown), which may include at least one of a keyboard, a mouse and/or an input pad, for receiving input from a user.

The storage unit 1 is configured to store multiple pieces of training answer information that are respectively related to multiple respondents and that are related to a sleep-related questionnaire. The sleep-related questionnaire includes multiple questions that are each related to at least one of multiple evaluation subjects, wherein the multiple evaluation subjects include a particular evaluation subject. Each of the pieces of training answer information includes multiple answers which are respectively related to the questions of the sleep-related questionnaire, and hence each of the answers is related to at least one of the multiple evaluation subjects. Each of the pieces of training answer information is associated with a classification result that is one of insomnia and sleep apnea. In some embodiments, the sleep-related questionnaire is the Pittsburgh Sleep Quality Index (PSQI), the evaluation subjects are the seven components of the PSQI that include subjective sleep quality, sleep latency, sleep duration, habitual sleep efficiency, sleep disturbances, use of sleeping medication and daytime dysfunction, and the particular evaluation subject is sleep disturbances.

The storage unit 1 is further configured to store multiple pieces of training blood oxygen level information that are respectively related to blood oxygen levels of a plurality of respondents during nighttime sleep. The pieces of training blood oxygen level information are each associated with a sleep apnea level that is one of low, medium and high.

The storage unit 1 is further configured to store multiple training electrocardiogram (ECG) signals that are each related to electrical activity of the heart of a respective one of multiple respondents during nighttime sleep. Each of the training ECG signals is divided into multiple segmental signals, each of which is associated with a sleep stage label that is one of a wakefulness stage, a rapid eye movement (REM) stage and a non-rapid eye movement (NREM) stage. According to some embodiments, each training ECG signal may be divided based on a fixed time duration. For example, in an embodiment, each training ECG signal is divided based on a 120-second duration, so that the segmental signals each have a duration of 120 seconds. However, in other embodiments, the duration of each segmental signal may be shorter or longer than 120 seconds.

The computing device is configured to perform a method for establishing at least one sleep-related evaluation model. The method for establishing at least one sleep-related evaluation model includes a first procedure for establishing a sleep evaluation model, a second procedure for establishing a sleep apnea evaluation model, and a third procedure for establishing a sleep stage determination model.

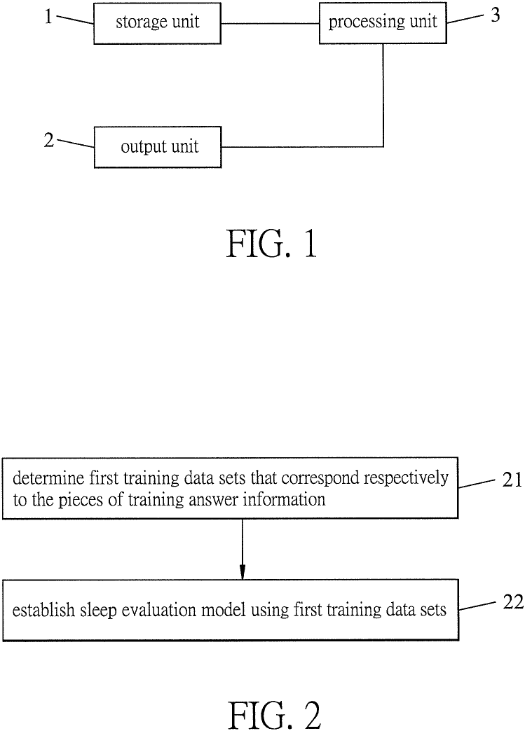

FIG. 2 exemplarily illustrates the first procedure according to an embodiment of the disclosure. Referring to FIG. 2, the first procedure includes Steps 21 and 22. In Step 21, the processing unit 3 determines multiple first training data sets that correspond respectively to the pieces of training answer information stored in the storage unit 1. In Step 22, the processing unit 3 uses the first training data sets determined in Step 21 to train a first machine learning model, in order to establish a sleep evaluation model. According to some embodiments, each of the first machine learning model and the sleep evaluation model may be a deep neural network (DNN) model. The sleep evaluation model thus established is stored into the storage unit 1.

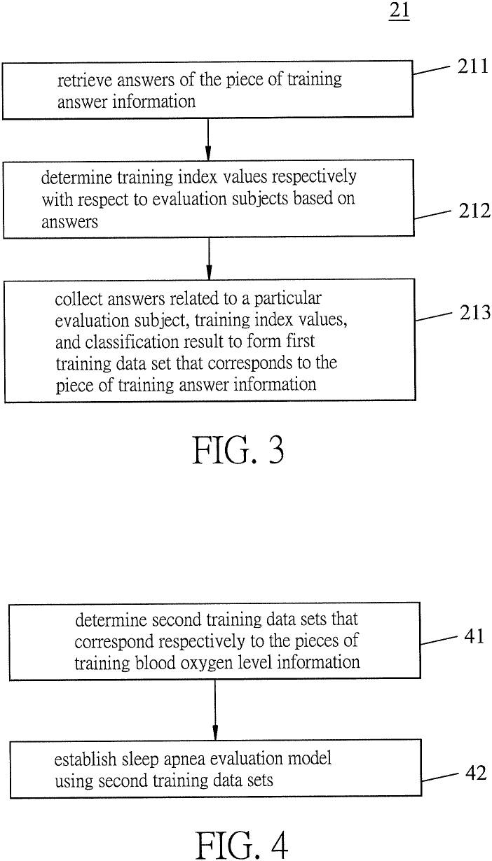

Step 21 includes Sub-steps 211-213 that are illustrated in FIG. 3 and that are to be performed with respect to each of the pieces of training answer information. Referring to FIG. 3, in Sub-step 211, the processing unit 3 retrieves the answers of the piece of training answer information from the storage unit 1.

In Sub-step 212, the processing unit 3 determines, with respect to each of the multiple evaluation subjects, a training index value based on those of the answers that are related to the evaluation subject (namely, one or more of the answers that are related to the evaluation subject). In embodiments where the sleep-related questionnaire is the PSQI, the training index value is the component score calculated for the evaluation subject.

In Sub-step 213, the processing unit 3 collects the training index values thus determined for the multiple evaluation subjects, the classification result associated with the piece of training answer information, and those of the answers that are related to the particular evaluation subject to form the first training data set that corresponds to the piece of training answer information.

FIG. 4 exemplarily illustrates the second procedure according to an embodiment of the disclosure. Referring to FIG. 4, the second procedure includes Steps 41 and 42. In Step 41, the processing unit 3 determines multiple second training data sets that correspond respectively to the pieces of training blood oxygen level information stored in the storage unit 1. In Step 42, the processing unit 3 uses the second training data sets determined in Step 41 to train a second machine learning model, in order to establish a sleep apnea evaluation model. According to some embodiments, each of the second machine learning model and the sleep apnea evaluation model may be a DNN model. The sleep apnea evaluation model thus established is stored into the storage unit 1.

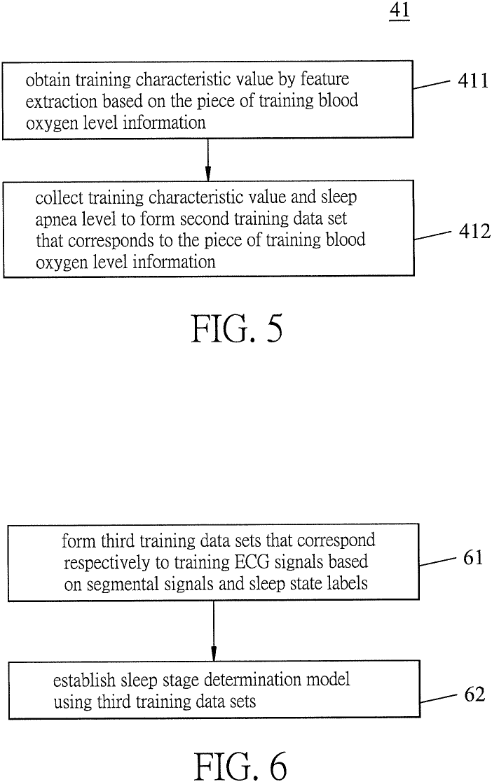

Step 41 includes Sub-steps 411-412 that are illustrated in FIG. 5 and that are to be performed with respect to each of the pieces of training blood oxygen level information. Referring to FIG. 5, in Sub-step 411, the processing unit 3 obtains a training characteristic value by feature extraction based on the piece of training blood oxygen level information. According to some embodiments, the training characteristic value may be obtained by applying the method disclosed in “Screening for Sleep Apnea Using Pulse Oximetry and A Clinical Score” by Adrian J. Williams et al. on the piece of training blood oxygen level information. Next, in Sub-step 412, the processing unit 3 collects the training characteristic value and the sleep apnea level associated with the piece of training blood oxygen level information to form the second training data set that corresponds to the piece of training blood oxygen level information.

FIG. 6 exemplarily illustrates the third procedure according to an embodiment of the disclosure. Referring to FIG. 6, the third procedure includes Steps 61 and 62. In Step 61, the processing unit 3 collects, with respect to each of the training ECG signals stored in the storage unit 1, the segmental signals of the training ECG signal and the sleep state labels respectively associated with the segmental signals, to form a third training data set that corresponds to the training ECG signal. In Step 62, the processing unit 3 uses the third training data sets thus obtained for the training ECG signals in Step 61 to train a third machine learning model, in order to establish a sleep stage determination model. According to some embodiments, each of the third machine learning model and the sleep stage determination model may be a DNN model. The sleep stage determination model thus established is stored into the storage unit 1.

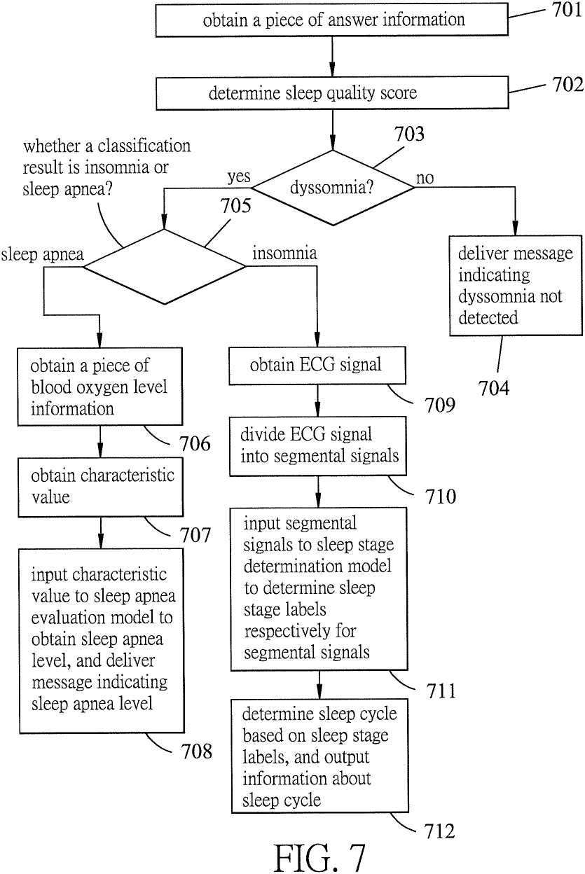

The computing device is further configured to perform a method for sleep evaluation. FIG. 7 exemplarily illustrates the method according to an embodiment of the disclosure. Referring to FIG. 7, the method includes Steps 701-712.

In Step 701, the processing unit 3 obtains a piece of answer information that is related to a respondent and the sleep-related questionnaire which includes the multiple questions. The piece of answer information includes multiple answers that are respectively related to the questions, and that are each related to at least one of the multiple evaluation subjects which include the particular evaluation subject. According to some embodiments, the processing unit 3 may obtain the piece of answer information through an input unit of the computing device that is operated by the respondent for answering the sleep-related questionnaire. The processing unit 3 may alternatively obtain the piece of answer information by reading the piece of answer information from a portable storage medium (e.g., a thumb drive or a compact disk), or receiving the piece of answer information that is sent by another computing device.

In Step 702, the processing unit 3 determines a sleep quality score based on the piece of answer information thus obtained in Step 701.

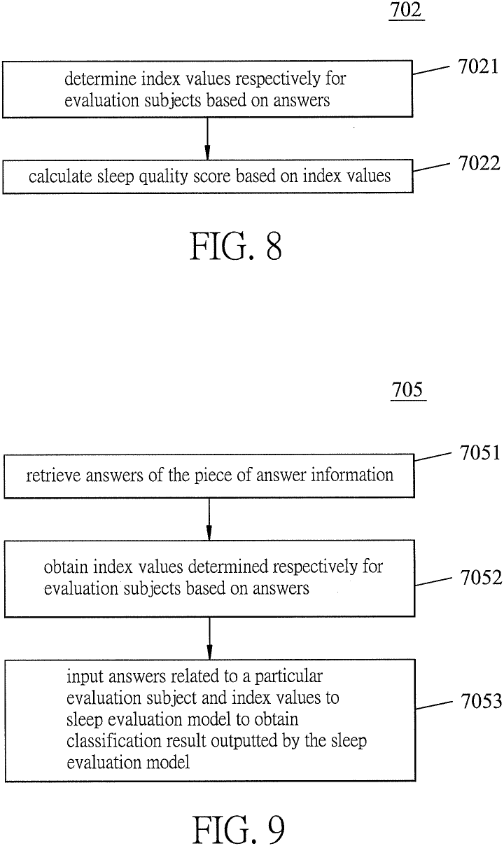

Step 702 includes Sub-steps 7021-7022 that are illustrated in FIG. 8. Referring to FIG. 8, in Sub-step 7021, the processing unit 3 determines, with respect to each of the multiple evaluation subjects, an index value based on those of the answers that are related to the evaluation subject. In Sub-step 7022, the processing unit 3 calculates the sleep quality score based on the index values thus determined for the multiple evaluation subjects. In embodiments where the sleep-related questionnaire is the PSQI, the index values are the component scores calculated respectively for the evaluation subjects, and the sleep quality score is the global PSQI score which is a sum of the seven component scores.

Returning to FIG. 7, in Step 703, the processing unit 3 determines whether dyssomnia is detected with respect to the respondent based on the sleep quality score thus determined. If so, the process goes to Step 705; otherwise, the process goes to Step 704. According to some embodiments, whether dyssomnia is detected may be determined by comparing the sleep quality score with a threshold score, and dyssomnia may be detected when the sleep quality score exceeds the threshold score. In embodiments where the sleep-related questionnaire is the PSQI, dyssomnia is detected when it is found that the sleep quality score exceeds 5 points.

In Step 704, the processing unit 3 controls the output unit 2 to deliver, to a user of the computing device, a message indicating that dyssomnia is not detected with respect to the respondent (e.g., by showing the message on the output unit 2 in cases where the output unit 2 is a display).

In Step 705, the processing unit 3 uses the sleep evaluation model stored in the storage unit 1 to determine a classification result with respect to the respondent based on the piece of answer information, and controls the output unit 2 to deliver another message indicating the classification result to the user. The classification result is either insomnia or sleep apnea. If the classification result thus determined is sleep apnea, the process goes to Step 706; and if the classification result thus determined is insomnia, the process goes to Step 709.

Step 705 includes Sub-steps 7051-7053 that are illustrated in FIG. 9. Referring to FIG. 9, in Sub-step 7051, the processing unit 3 retrieves the answers of the piece of answer information. In Sub-step 7052, the processing unit 3 obtains, with respect to each of the multiple evaluation subjects, the index value that was determined (in Sub-step 7021) based on those of the answers that are related to the evaluation subject. In Sub-step 7053, the processing unit 3 inputs the index values thus determined for the multiple evaluation subjects and those of the answers that are related to the particular evaluation subject to the sleep evaluation model, so that the sleep evaluation model outputs the classification result associated with the respondent.

Returning to FIG. 7, in Step 706, the processing unit 3 obtains a piece of blood oxygen level information that is related to a blood oxygen level of the respondent during nighttime sleep. In some embodiments, the piece of blood oxygen level information is pre-stored in the storage unit 1. In some other embodiments, the processing unit 3 obtains the piece of blood oxygen level information by reading the piece of blood oxygen level information from a portable storage medium, or receiving the piece of blood oxygen level information from another computing device.

In Step 707, the processing unit 3 obtains a characteristic value by feature extraction (in the same manner as with Sub-step 411) based on the piece of blood oxygen level information.

In Step 708, the processing unit 3 inputs the characteristic value thus obtained to the sleep apnea evaluation model stored in the storage unit 1, so that the sleep apnea evaluation model outputs a sleep apnea level that is one of low, medium and high. The processing unit 3 also controls the output unit 2 to deliver another message indicating the sleep apnea level to the user.

In Step 709, the processing unit 3 obtains an ECG signal that is related to nighttime sleep of the respondent. In some embodiments, the ECG signal is pre-stored in the storage unit 1. In some other embodiments, the processing unit 3 obtains the ECG signal by reading the ECG signal from a portable storage medium, or receiving the ECG signal from another computing device.

In Step 710, the processing unit 3 divides the ECG signal into multiple segmental signals based on the fixed time duration, based on which the training ECG signals were divided.

In Step 711, the processing unit 3 inputs the segmental signals of the ECG signal to the sleep stage determination model, in order to determine, for each of the segmental signals, a sleep stage label associated with the segmental signal, which is one of a wakefulness stage, a REM stage and a NREM stage.

In Step 712, the processing unit 3 determines a sleep cycle based on the sleep stage labels thus determined for the segmental signals, and controls the output unit 2 to output information about the sleep cycle thus determined to the user. In some embodiments, the output unit 2 outputs the information about the sleep cycle by showing a hypnogram on a display.

It is noted that the method for sleep evaluation and the method for establishing at least one sleep-related evaluation model are not necessarily to be performed by the same computing device. That is, a first computing device may first establish the sleep evaluation model, the sleep apnea evaluation model and the sleep stage determination model by performing the method for establishing at least one sleep-related evaluation model, and then send, directly or indirectly, said models to a second computing device, so that the second computing device may perform the method for sleep evaluation, in which these models are used.

Through the methods and the computing device disclosed herein, the present disclosure provides an effective and economical (in terms of both of time and human resources) way to assist physicians in diagnosing sleep disorders including insomnia and sleep apnea.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for establishing at least one sleep-related evaluation model that is to be performed by a computing device, the computing device storing multiple pieces of training answer information that are respectively related to multiple respondents and that are related to a sleep-related questionnaire including multiple questions, each of the pieces of training answer information including multiple answers that are respectively related to the questions and that are each related to at least one of multiple evaluation subjects, the multiple evaluation subjects including a particular evaluation subject, each of the pieces of training answer information being associated with a classification result that is one of insomnia and sleep apnea, the method comprising steps of:
determining multiple first training data sets that correspond respectively to the pieces of training answer information by, with respect to each of the pieces of training answer information,
retrieving the answers of the piece of training answer information,
with respect to each of the multiple evaluation subjects, determining a training index value based on those of the answers that are related to the evaluation subject, and
collecting the training index values thus determined for the multiple evaluation subjects, the classification result associated with the piece of training answer information, and those of the answers that are related to the particular evaluation subject to form the first training data set that corresponds to the piece of training answer information; and
using the first training data sets to train a machine learning model, in order to establish a sleep evaluation model.

2. The method of claim 1, wherein the sleep-related questionnaire is the Pittsburgh Sleep Quality Index (PSQI), and the particular evaluation subject is sleep disturbances.

3. The method of claim 1, the computing device further storing multiple pieces of training blood oxygen level information that are respectively related to blood oxygen levels of a plurality of respondents during nighttime sleep, each of the pieces of training blood oxygen level information being associated with a sleep apnea level that is one of low, medium and high, the method further comprising steps of:
determining multiple second training data sets that correspond respectively to the pieces of training blood oxygen level information by, with respect to each of the pieces of training blood oxygen level information,
obtaining a training characteristic value by feature extraction based on the piece of training blood oxygen level information, and
collecting the training characteristic value and the sleep apnea level associated with the piece of training blood oxygen level information to form the second training data set that corresponds to the piece of training blood oxygen level information; and
using the second training data sets to train another machine learning model, in order to establish a sleep apnea evaluation model.

4. The method of claim 1, the computing device further storing multiple training electrocardiogram (ECG) signals that are each related to electrical activity of the heart of a respective one of multiple respondents during nighttime sleep, each of the training ECG signals being divided into multiple segmental signals, each of which is associated with a sleep stage label that is one of a wakefulness stage, a rapid eye movement (REM) stage and a non-rapid eye movement (NREM) stage, the method further comprising steps of:
with respect to each of the training ECG signals, collecting the segmental signals of the training ECG signal and the sleep state labels respectively associated with the segmental signals to form a third training data set that corresponds to the training ECG signal; and
using the third training data sets thus obtained for the training ECG signals to train another machine learning model, in order to establish a sleep stage determination model.

5. A method for sleep evaluation that is to be performed by a computing device, the computing device storing a sleep evaluation model that is established according to the method of claim 1, the method for sleep evaluation comprising steps of:
obtaining a piece of answer information that is related to a respondent and a sleep-related questionnaire, wherein the sleep-related questionnaire includes multiple questions, and the piece of answer information includes multiple answers respectively related to the questions;
determining a sleep quality score based on the piece of answer information thus obtained;
determining whether dyssomnia is detected based on the sleep quality score thus determined; and
when dyssomnia is detected, using the sleep evaluation model to determine a classification result with respect to the respondent based on the piece of answer information, wherein the classification result is one of insomnia and sleep apnea.

6. The method for sleep evaluation of claim 5, wherein the answers in the piece of answer information are each related to at least one of multiple evaluation subjects, the multiple evaluation subjects include a particular evaluation subject, and the step of using the sleep evaluation model includes sub-steps of:
retrieving the answers of the piece of answer information;
with respect to each of the multiple evaluation subjects, obtaining an index value that is determined based on those of the answers that are related to the evaluation subject; and
inputting the index values thus determined for the multiple evaluation subjects and those of the answers that are related to the particular evaluation subject to the sleep evaluation model, so that the sleep evaluation model outputs the classification result associated with the respondent.

7. The method for sleep evaluation of claim 5, wherein the answers in the piece of answer information are each related to at least one of multiple evaluation subjects, the multiple evaluation subjects include a particular evaluation subject, and the step of determining a sleep quality score includes sub-steps of:
with respect to each of the multiple evaluation subjects, determining an index value based on those of the answers that are related to the evaluation subject; and
calculating the sleep quality score based on the index values thus determined for the multiple evaluation subjects.

8. The method for sleep evaluation of claim 5, the computing device further storing multiple pieces of training blood oxygen level information that are respectively related to blood oxygen levels of a plurality of respondents during nighttime sleep, each of the pieces of training blood oxygen level information being associated with a sleep apnea level that is one of low, medium and high, the method further comprising steps of:
determining multiple second training data sets that correspond respectively to the pieces of training blood oxygen level information by, with respect to each of the pieces of training blood oxygen level information,
obtaining a training characteristic value by feature extraction based on the piece of training blood oxygen level information, and
collecting the training characteristic value and the sleep apnea level associated with the piece of training blood oxygen level information to form the second training data set that corresponds to the piece of training blood oxygen level information; and
using the second training data sets to train another machine learning model, in order to establish a sleep apnea evaluation model,
the method for sleep evaluation further comprising following steps that are to be performed when the classification result is sleep apnea:
obtaining a piece of blood oxygen level information that is related to a blood oxygen level of the respondent during nighttime sleep;
obtaining a characteristic value by feature extraction based on the piece of blood oxygen level information; and
inputting the characteristic value thus obtained to the sleep apnea evaluation model, so that the sleep apnea evaluation model outputs a sleep apnea level that is one of low, medium and high.

9. The method for sleep evaluation of claim 5 the computing device further storing multiple training electrocardiogram (ECG) signals that are each related to electrical activity of the heart of a respective one of multiple respondents during nighttime sleep, each of the training ECG signals being divided into multiple segmental signals, each of which is associated with a sleep stage label that is one of a wakefulness stage, a rapid eye movement (REM) stage and a non-rapid eye movement (NREM) stage, the method further comprising steps of:
with respect to each of the training ECG signals, collecting the segmental signals of the training ECG signal and the sleep state labels respectively associated with the segmental signals to form a third training data set that corresponds to the training ECG signal; and
using the third training data sets thus obtained for the training ECG signals to train another machine learning model, in order to establish a sleep stage determination model,
the method for sleep evaluation further comprising following steps that are to be performed when the classification result is insomnia:
obtaining an electrocardiogram (ECG) signal that is related to electrical activity of the heart of the respondent during nighttime sleep;
dividing the ECG signal into multiple segmental signals; and
inputting the segmental signals to the sleep stage determination model, in order to determine, for each of the segmental signals, a sleep stage label associated with the segmental signal, which is one of a wakefulness stage, a rapid eye movement (REM) stage and a non-rapid eye movement (NREM) stage.

10. A computing device, comprising:
a storage unit storing multiple pieces of training answer information that are respectively related to multiple respondents and that are related to a sleep-related questionnaire including multiple questions, wherein each of the pieces of training answer information includes multiple answers that are respectively related to the questions and that are each related to at least one of multiple evaluation subjects, the multiple evaluation subjects include a particular evaluation subject, and each of the pieces of training answer information is associated with a classification result that is one of insomnia and sleep apnea; and
a processing unit electrically connected to said storage unit, and configured to:
determine multiple first training data sets that correspond respectively to the pieces of training answer information stored in said storage unit by, with respect to each of the pieces of training answer information,
retrieving the answers of the piece of training answer information,
with respect to each of the multiple evaluation subjects, determining a training index value based on those of the answers that are related to the evaluation subject, and
collecting the training index values thus determined for the multiple evaluation subjects, the classification result associated with the piece of training answer information, and those of the answers that are related to the particular evaluation subject to form the first training data set that corresponds to the piece of training answer information; and
use the first training data sets to train a machine learning model, in order to establish a sleep evaluation model.

11. The computing device of claim 10, wherein the sleep-related questionnaire is the Pittsburgh Sleep Quality Index (PSQI), and the particular evaluation subject is sleep disturbances.

12. The computing device of claim 10, wherein:
said storage unit further stores multiple pieces of training blood oxygen level information that are respectively related to blood oxygen levels of a plurality of respondents during nighttime sleep, wherein each of the pieces of training blood oxygen level information is associated with a sleep apnea level that is one of low, medium and high; and
said processing unit is further configured to:
determine multiple second training data sets that correspond respectively to the pieces of training blood oxygen level information stored in said storage unit by, with respect to each of the pieces of training blood oxygen level information,
obtaining a training characteristic value by feature extraction based on the piece of training blood oxygen level information, and
collecting the training characteristic value and the sleep apnea level associated with the piece of training blood oxygen level information to form the second training data set that corresponds to the piece of training blood oxygen level information; and
use the second training data sets to train another machine learning model, in order to establish a sleep apnea evaluation model.

13. The computing device of claim 10, wherein:
said storage unit further stores multiple training electrocardiogram (ECG) signals that are each related to electrical activity of the heart of a respective one of multiple respondents during nighttime sleep, wherein each of the training ECG signals is divided into multiple segmental signals, each of which is associated with a sleep stage label that is one of a wakefulness stage, a rapid eye movement (REM) stage and a non-rapid eye movement (NREM) stage; and
said processing unit is further configured to:
with respect to each of the training ECG signals stored in said storage unit, collect the segmental signals of the training ECG signal and the sleep state labels respectively associated with the segmental signals to form a third training data set that corresponds to the training ECG signal; and
use the third training data sets thus obtained for the training ECG signals to train another machine learning model, in order to establish a sleep stage determination model.

14. The computing device of claim 10, wherein said processing unit is further configured to:
obtain a piece of answer information that is related to a respondent and the sleep-related questionnaire, wherein the piece of answer information includes multiple answers respectively related to the questions of the sleep-related questionnaire;
determine a sleep quality score based on the piece of answer information thus obtained;
determine whether dyssomnia is detected based on the sleep quality score thus determined; and
when dyssomnia is detected, use the sleep evaluation model to determine a classification result with respect to the respondent based on the piece of answer information, wherein the classification result is one of insomnia and sleep apnea.

15. The computing device of claim 14, wherein:
the answers in the piece of answer information are each related to at least one of the multiple evaluation subjects, wherein the multiple evaluation subjects include the particular evaluation subject; and
said processing unit is configured to use the sleep evaluation model to determine the classification result by:
retrieving the answers of the piece of answer information;
with respect to each of the multiple evaluation subjects, obtaining an index value that is determined based on those of the answers that are related to the evaluation subject; and
inputting the index values thus determined for the multiple evaluation subjects and those of the answers that are related to the particular evaluation subject to the sleep evaluation model, so that the sleep evaluation model outputs the classification result associated with the respondent.

16. The computing device of claim 14, wherein:
the answers in the piece of answer information are each related to at least one of the multiple evaluation subjects, wherein the multiple evaluation subjects include the particular evaluation subject; and
said processing unit is configured to determine the sleep quality score by:
with respect to each of the multiple evaluation subjects, determining an index value based on those of the answers that are related to the evaluation subject; and
calculating the sleep quality score based on the index values thus determined for the multiple evaluation subjects.

17. The computing device of claim 14, wherein:
said storage unit further stores multiple pieces of training blood oxygen level information that are respectively related to blood oxygen levels of a plurality of respondents during nighttime sleep, wherein each of the pieces of training blood oxygen level information is associated with a sleep apnea level that is one of low, medium and high;
said processing unit is configured to:
determine multiple second training data sets that correspond respectively to the pieces of training blood oxygen level information stored in said storage unit by, with respect to each of the pieces of training blood oxygen level information,
obtaining a training characteristic value by feature extraction based on the piece of training blood oxygen level information, and
collecting the training characteristic value and the sleep apnea level associated with the piece of training blood oxygen level information to form the second training data set that corresponds to the piece of training blood oxygen level information;
use the second training data sets to train another machine learning model, in order to establish a sleep apnea evaluation model; and
said processing unit is further configured to, when it is determined that the classification result associated with the respondent is sleep apnea,
obtain a piece of blood oxygen level information that is related to a blood oxygen level of the respondent during nighttime sleep;
obtain a characteristic value by feature extraction based on the piece of blood oxygen level information; and
input the characteristic value thus obtained to the sleep apnea evaluation model, so that the sleep apnea evaluation model outputs a sleep apnea level that is one of low, medium and high.

18. The computing device of claim 14, wherein:
said storage unit further stores multiple training electrocardiogram (ECG) signals that are each related to electrical activity of the heart of a respective one of multiple respondents during nighttime sleep, wherein each of the training ECG signals is divided into multiple segmental signals, each of which is associated with a sleep stage label that is one of a wakefulness stage, a rapid eye movement (REM) stage and a non-rapid eye movement (NREM) stage;
said processing unit is configured to:
with respect to each of the training ECG signals stored in said storage unit, collect the segmental signals of the training ECG signal and the sleep state labels respectively associated with the segmental signals to form a third training data set that corresponds to the training ECG signal, and
use the third training data sets thus obtained for the training ECG signals to train another machine learning model, in order to establish a sleep stage determination model; and
said processing unit is further configured to, when it is determined that the classification result associated with the respondent is insomnia,
obtain an ECG signal that is related to electrical activity of the heart of the respondent during nighttime sleep,
divide the ECG signal into multiple segmental signals, and
input the segmental signals to the sleep stage determination model, in order to determine, for each of the segmental signals of the ECG signal, a sleep stage label associated with the segmental signal, which is one of the wakefulness stage, the REM stage and the NREM stage.
